(19) United States
(12) Patent Application Publication
(10) Pub. No.: US 2025/0099051 A1
(43) Pub. Date: Mar. 27, 2025

(54) APPARATUS FOR IMAGING SYSTEM, X-RAY FILTERING APPARATUS, AND IMAGING SYSTEM

(71) Applicant: GE Precision Healthcare LLC, Waukesha, WI (US)

(72) Inventors: Xueli Wang, Beijing (CN); Dan Liu, Beijing (CN); Yuwen Dong, Beijing (CN); Liya Cao, Beijing (CN)

(21) Appl. No.: 18/891,997

(22) Filed: Sep. 20, 2024

(30) Foreign Application Priority Data
Sep. 22, 2023 (CN) 202311240014.6

(51) Int. Cl. A61B 6/40

(52) U.S. Cl. CPC A61B 6/4035; A61B 6/4078

(57) ABSTRACT

The present disclosure relates to an apparatus for an imaging system, a X-ray filtering apparatus, and an imaging system. The apparatus includes: a first filter assembly, including at least one filter and located on one side of the path of a radiation beam; a second filter assembly, including at least one filter and located on the other side of the path of the radiation beam; and a driving system, used for moving the first filter assembly and the second filter assembly relative to each other to move one filter in the first filter assembly and/or one filter in the second filter assembly into the path of the radiation beam; wherein in a path direction of the radiation beam, the first filter assembly includes at least a first layer of filters and a second layer of filters located at different heights, and the second filter assembly includes at least a third layer of filters, the third layer of filters being located at a different height than the first layer of filters or the second layer of filters.

### CROSS-REFERENCE TO RELATED APPLICATIONS

This application claims priority to Chinese Application No. 202311240014.6, filed on Sep. 22, 2023, the disclosure of which is incorporated herein by reference in its entirety.

### TECHNICAL FIELD

The present invention relates to the field of medical imaging, and relates in particular to an apparatus for an imaging system, an X-ray filtering apparatus, and an imaging system.

### BACKGROUND

A non-invasive imaging modality may transmit energy in the form of radiation into an imaging subject. On the basis of the transmitted energy, an image indicating structural or functional information of the interior of the imaging subject can then be generated. In computed tomography (CT) imaging, radiation is transmitted from a radiation source through the imaging subject to a detector. An X-ray filter may be positioned between the radiation source and the imaging subject to adjust the spatial distribution of the radiant energy on the basis of an anatomical structure of the imaging subject. The X-ray filter may be designed to distribute high radiant energy to a particular imaging region of the subject. Therefore, the amplitude of a signal received by the imaging detector is improved, and the radiation dose in the periphery of the particular imaging subject is reduced. A bowtie filter is a commonly used X-ray filter, and different bowtie filters may be required for different subjects and different anatomical structures of the same subject. For example, different shapes and sizes of bowtie filters may be designed to image subjects having different body sizes or different regions (such as the head, chest, and abdomen) of the subject's body. To adapt to different scanning requirements, the imaging system needs to be configured with a plurality of different types of X-ray filters, but an excessive number of X-ray filters will make the overall size of the filter apparatus large and occupy a great deal of space. Therefore, filter combinations may be used to meet the requirements. For example, a bowtie filter may be designed for a subject having a large body size, and then different shaping or hardening filters and the bowtie filter may be combined to adjust the beam, so as to perform scanning and imaging of subjects having different body type or different body parts of the same subject. Furthermore, different functional filters may need to be individually positioned between the radiation source and the detector to adapt to different clinical purposes or to calibrate the imaging system.

Therefore, there is a need for a new design of a filter apparatus for integrating a plurality of filters to achieve a variety of X-ray filtering functions.

### SUMMARY

It is an object of the present invention to overcome the above and/or other problems in the prior art, and provided thereby is a new design of an X-ray filtering apparatus, which can achieve a variety of X-ray filtering functions by using filter assemblies including multiple layers of filters that are arranged opposite to each other on two sides of a path of a radiation beam.

According to a first aspect of the present invention, an apparatus for an imaging system is provided. The apparatus includes a first filter assembly having at least one filter and being located on one side of the path of a radiation beam, a second filter assembly having at least one filter and being located on the other side of the path of the radiation beam, and a driving system used for moving the first filter assembly and the second filter assembly relative to each other to move one filter in the first filter assembly and/or one filter in the second filter assembly into the path of the radiation beam. In a path direction of the radiation beam, the first filter assembly includes at least a first layer of filters and a second layer of filters located at different heights, and the second filter assembly includes at least a third layer of filters, the third layer of filters being located at a different height than the first layer of filters.

Preferably, the third layer of filters includes a bowtie filter, and the bowtie filter is adapted to filter the radiation beam when imaging a subject having a small body size or a child's head.

Preferably, the first layer of filters includes at least a first filter having a first filtering characteristic and a second filter having a second filtering characteristic, the second layer of filters includes at least a third filter having a third filtering characteristic, and the third layer of filters includes at least a fourth filter having a fourth filtering characteristic.

Preferably, the first filter and the fourth filter are capable of being simultaneously moved into the path of the radiation beam to form a filter combination, wherein the fourth filter is a bowtie filter. Preferably, the first filter is an adult head filter such that the filter combination is adapted to filter the radiation beam when imaging an adult's head.

Preferably, the first filter is a shaping filter having a cross-shaped hollow portion, and the radiation beam passes through the cross-shaped hollow portion of the first filter without being attenuated.

Preferably, the fourth filter is adapted to filter the radiation beam when imaging a subject having a large body size.

Preferably, the second filter includes a transfer function correction filter or a blocking filter through which the radiation beam cannot penetrate.

Preferably, the first layer of filters and the second layer of filters do not overlap in a fan beam width direction of the radiation beam.

Preferably, the second filter assembly includes a fourth layer of filters, and the fourth layer of filters is located at a different height than the first layer of filters in the path direction of the radiation beam.

Preferably, the fourth layer of filters includes at least one filter that does not overlap with the third layer of filters in a fan beam width direction of the radiation beam.

Preferably, the third layer of filters further includes a fifth filter having a fifth filtering characteristic, and the fifth filter does not overlap with the fourth filter in the fan beam width direction of the radiation beam.

Preferably, the third filter and the fourth filter are capable of being simultaneously moved into the path of the radiation beam to form a filter combination.

Preferably, the third filter is adapted to filter the radiation beam when imaging a subject having a small body size or a child's head.

Preferably, the fourth filter is an adult head filter such that the filter combination is adapted to filter the radiation beam when imaging an adult's head.

Preferably, the fourth filter is a shaping filter having a cross-shaped hollow portion, and the radiation beam passes through the cross-shaped hollow portion of the first filter without being attenuated.

Preferably, the bowtie filter is adapted to filter the radiation beam when imaging a subject having a large body size.

According to a second aspect of the present invention an X-ray filtering apparatus for an imaging system is provided. The apparatus includes a first filter assembly having at least one filter and being located on one side of the path of a radiation beam, a second filter assembly having at least one filter and being located on the other side of the path of the radiation beam, and a driving system used for moving the first filter assembly and the second filter assembly relative to each other to move one filter in the first filter assembly and/or one filter in the second filter assembly into the path of the radiation beam. In a path direction of the radiation beam, the first filter assembly includes at least a first layer of filters and a second layer of filters located at different heights, and the second filter assembly includes at least a third layer of filters, the third layer of filters being located at a different height than the first layer of filters or the second layer of filters, the first layer of filters includes at least a first filter having a first filtering characteristic and a second filter having a second filtering characteristic, the second layer of filters includes at least a third filter having a third filtering characteristic, and the third layer of filters includes at least a fourth filter having a fourth filtering characteristic, and the fourth filter is capable of respectively forming a combined filter with at least one of the first filter, the second filter, or the third filter.

Preferably, the first layer of filters and the second layer of filters do not overlap in a fan beam width direction of the radiation beam.

Preferably, the first filter is a shaping filter having a cross-shaped hollow portion, and the radiation beam passes through the cross-shaped hollow portion of the first filter without being attenuated.

Preferably, the first filter and the second filter are mounted in the same plane, and the first filter is located between the second filter and the X-ray radiation path.

Preferably, the first filter is located in the path of the radiation beam together with the fourth filter to form a first combined filter.

Preferably, the fourth filter is a bowtie filter, and the first combined filter is adapted to filter the radiation beam when imaging an adult's head.

Preferably, the second filter assembly includes a fourth layer of filters, and the fourth layer of filters includes at least one sixth filter that does not overlap with the fourth filter in the fan beam width direction of the radiation beam.

Preferably, the sixth filter is capable of respectively forming a combined filter with at least one of the first filter, the second filter, or the third filter.

Preferably, the sixth filter is capable of being located in the path of the radiation beam together with the third filter to form a second combined filter.

According to a third aspect of the present invention, there is provided an imaging system performing an imaging examination on the basis of a scanning protocol. The imaging system includes a gantry used for receiving a scan subject, a radiation source positioned in the gantry for emitting a radiation beam, a detector positioned on an opposite surface of the gantry with respect to the radiation source, a motorized workbench used for moving the scan subject within the gantry, an X-ray filtering apparatus in accordance with this disclosure, and a controller configured to control the driving system on the basis of the scanning protocol such that a filter in the first filter assembly and/or the second filter assembly conforming to the scanning protocol is moved into the path of the radiation beam.

### BRIEF DESCRIPTION OF THE DRAWINGS

The present invention can be better understood by means of the description of the exemplary embodiments of the present invention in conjunction with the drawings, in which:

FIG. 1 shows an exemplary CT imaging system.

FIG. 2 shows an exemplary imaging system similar to the CT imaging system in

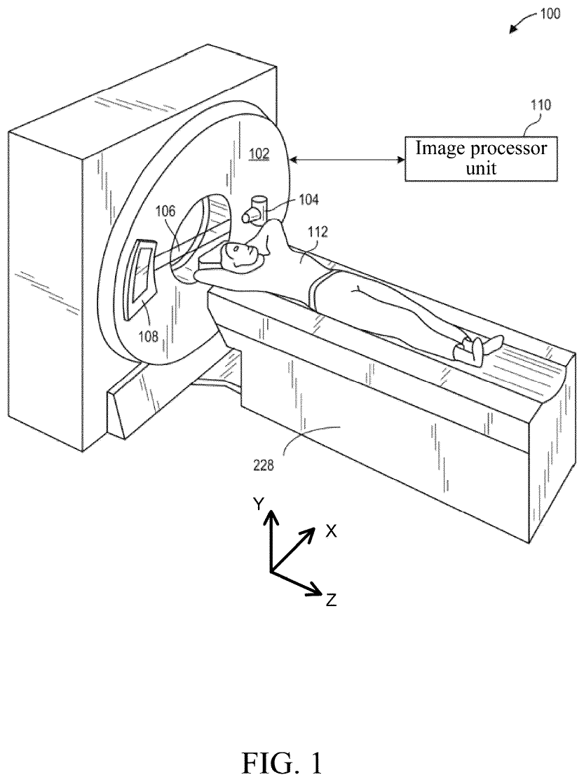

FIG. 1.

FIGS. 3A-3E are schematic block diagrams of an apparatus 300 for an imaging system according to a first exemplary embodiment of the present invention.

FIGS. 4A-4C are schematic block diagrams of an apparatus 400 for an imaging system according to a second exemplary embodiment of the present invention.

FIG. 5 is a schematic block diagram of an X-ray filtering apparatus 500 for an imaging system according to a third exemplary embodiment of the present invention.

FIG. 6 shows a schematic diagram of a shaping filter having a cross-shaped hollow portion.

FIG. 7 is an exemplary structure of an X-ray filtering apparatus 700 constructed according to the third exemplary embodiment of the present invention.

FIGS. 8-13 respectively show examples of a plurality of working positions of the X-ray filtering apparatus 700.

### DETAILED DESCRIPTION

Specific embodiments of the present invention will be described below. It should be noted that in the specific description of said embodiments, for the sake of brevity and conciseness, the present description cannot describe all of the features of the actual embodiments in detail. It should be understood that in the actual implementation process of any embodiment, just as in the process of any one engineering project or design project, a variety of specific decisions are often made to achieve specific goals of the developer and to meet system-related or business-related constraints, which may also vary from one embodiment to another.

Furthermore, it should also be understood that although efforts made in such development processes may be complex and tedious, for a person of ordinary skill in the art related to the content disclosed in the present invention, some design, manufacture, or production changes made on the basis of the technical content disclosed in the present disclosure are only common technical means, and should not be construed as the content of the present disclosure being insufficient.

Unless defined otherwise, technical terms or scientific terms used in the claims and description should have the usual meanings that are understood by those of ordinary skill in the technical field to which the present invention belongs. The terms “first,” “second,” and the like used in the description and claims of the patent application of the present invention do not denote any order, quantity, or importance, but are merely intended to distinguish between different constituents. The terms “one,” “a/an,” and the like do not denote a limitation of quantity, but rather denote the presence of at least one of the referenced items. The terms “include,” “comprise,” and the like are intended to mean that an element or article that appears before “include” or “comprise” encompasses elements or articles and equivalent elements that are listed after “include” or “comprise,” and do not exclude other elements or articles. The terms “connect,” “connected,” and the like are not limited to physical or mechanical connection, and are not limited to direct or indirect connection. The phrase “scan subject” generally includes, but is not limited to, a patient, an animal, or other subjects examined by a medical imaging device.

An apparatus for an imaging system provided according to an embodiment of the present disclosure will be described in detail below with reference to the accompanying drawings.

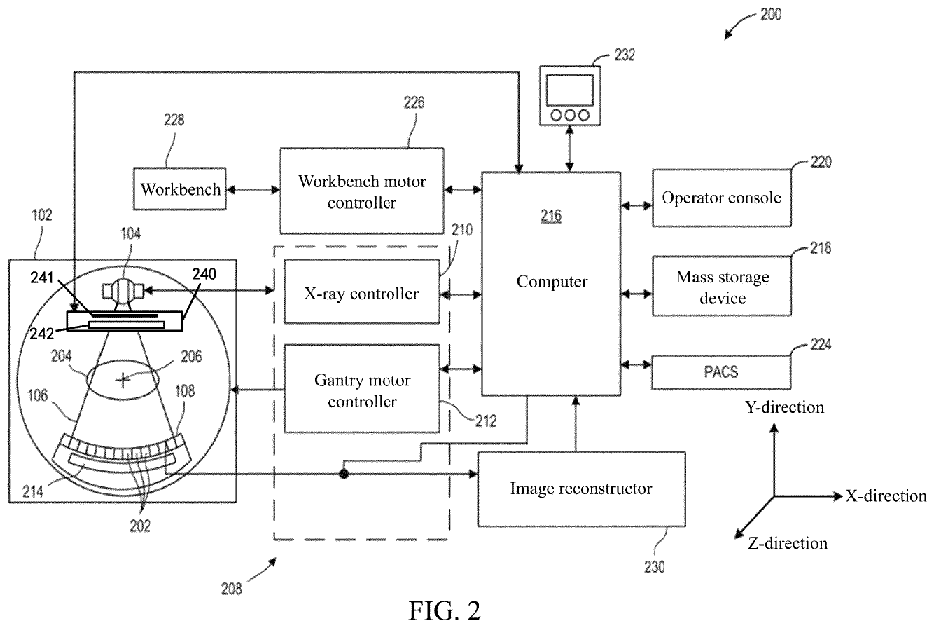

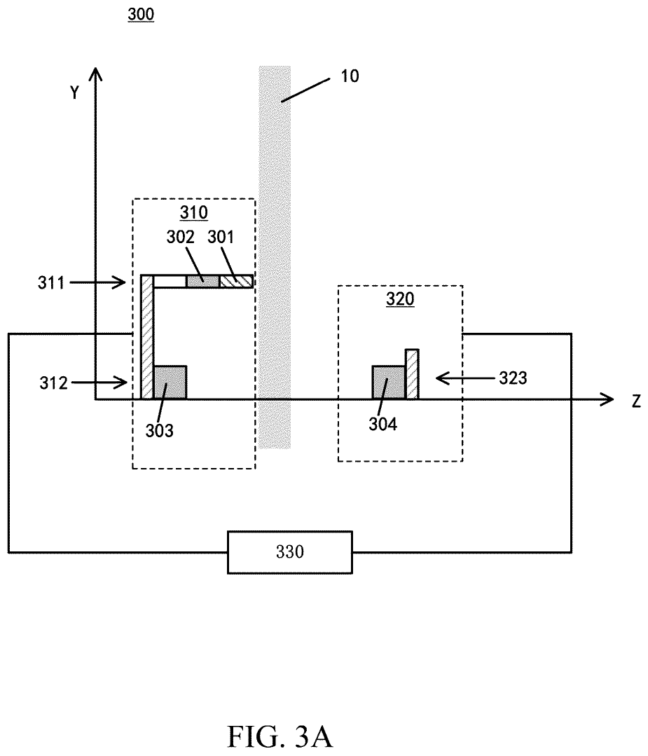

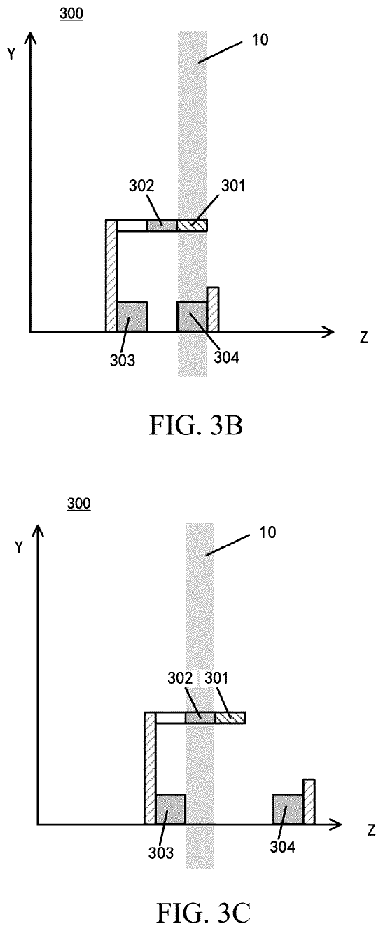

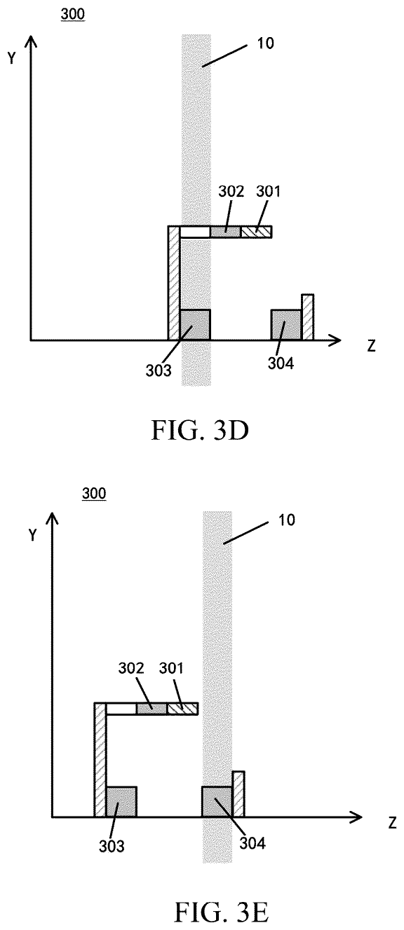

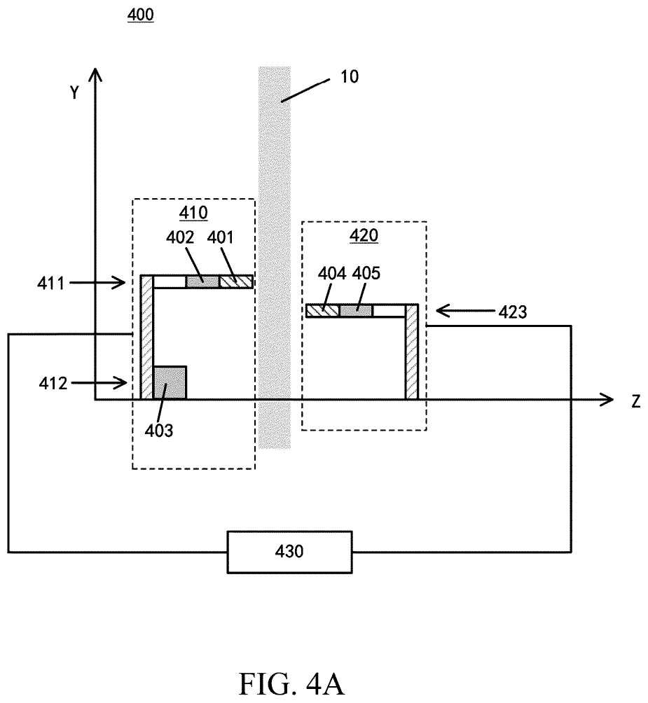

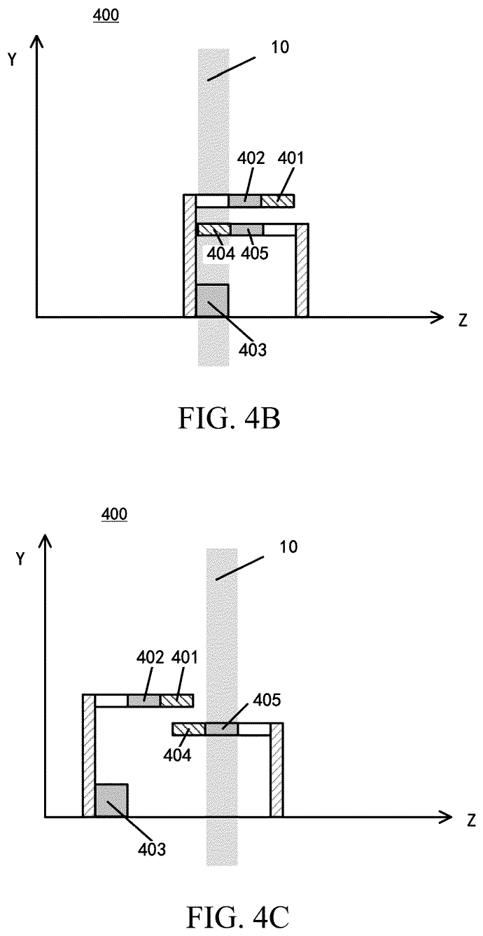

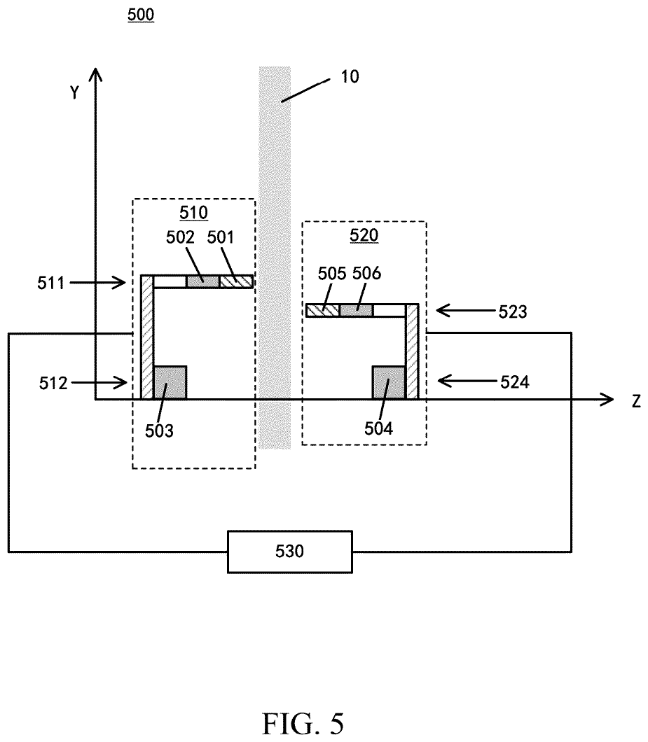

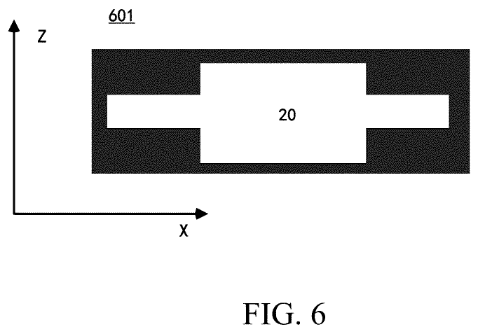

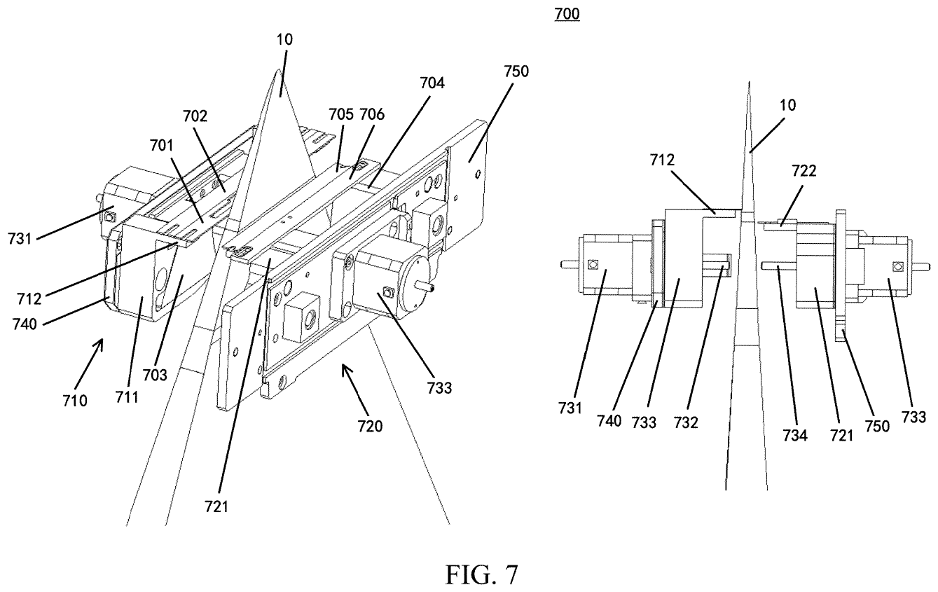

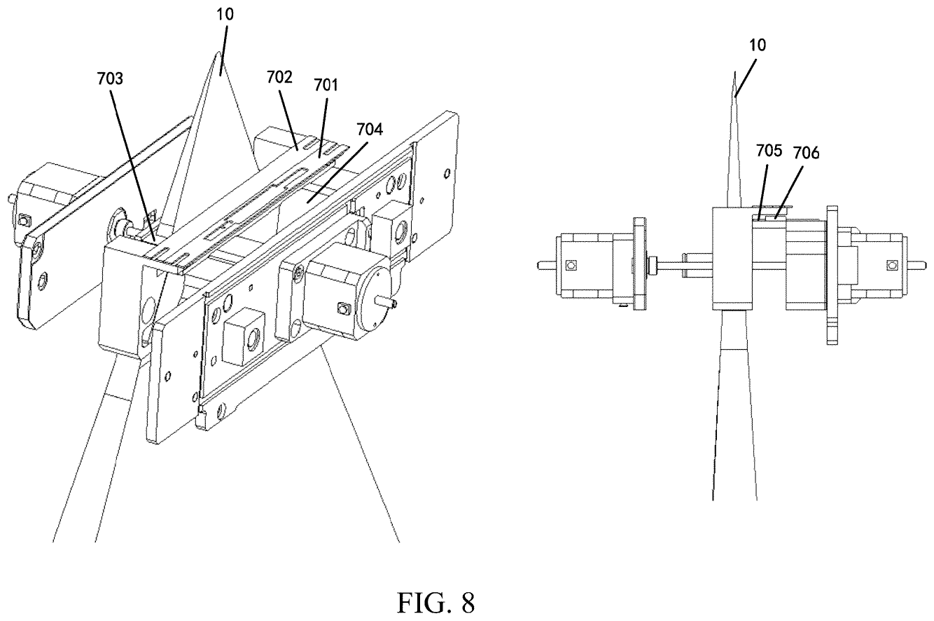

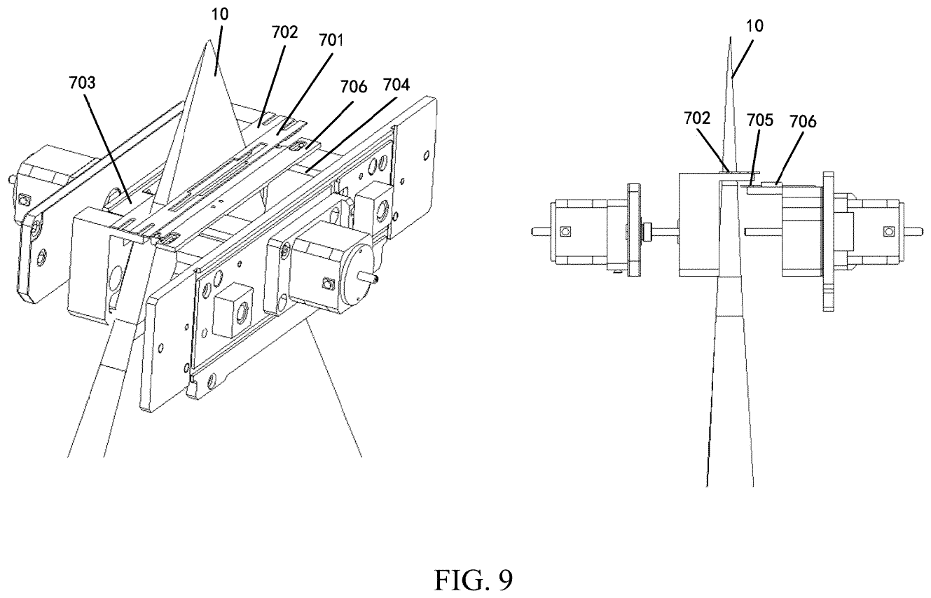

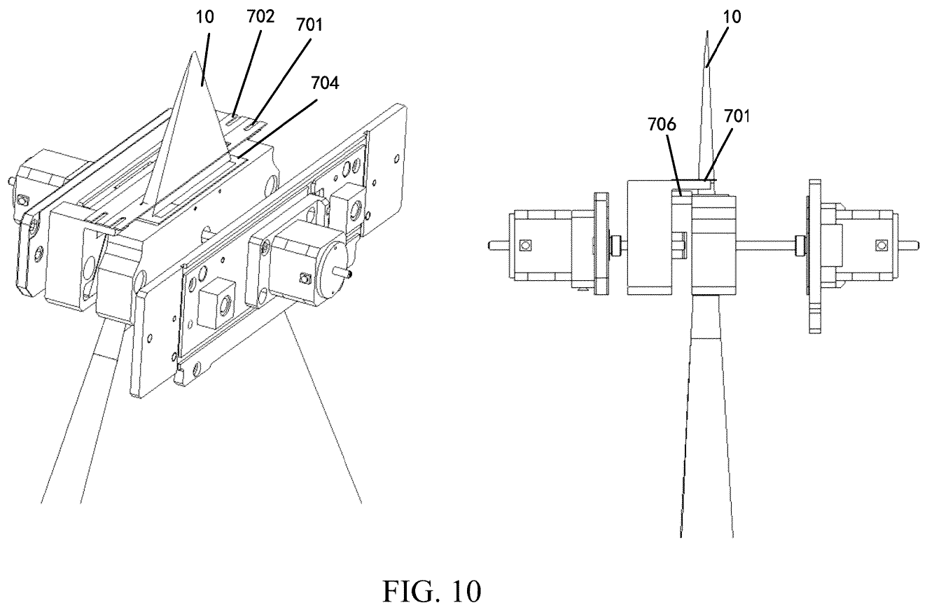

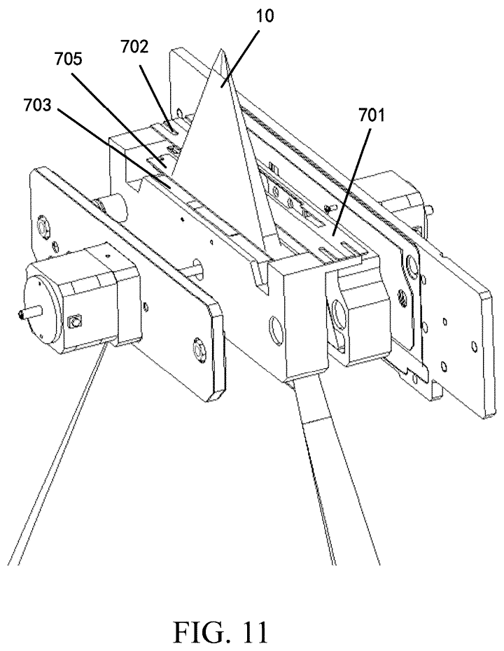

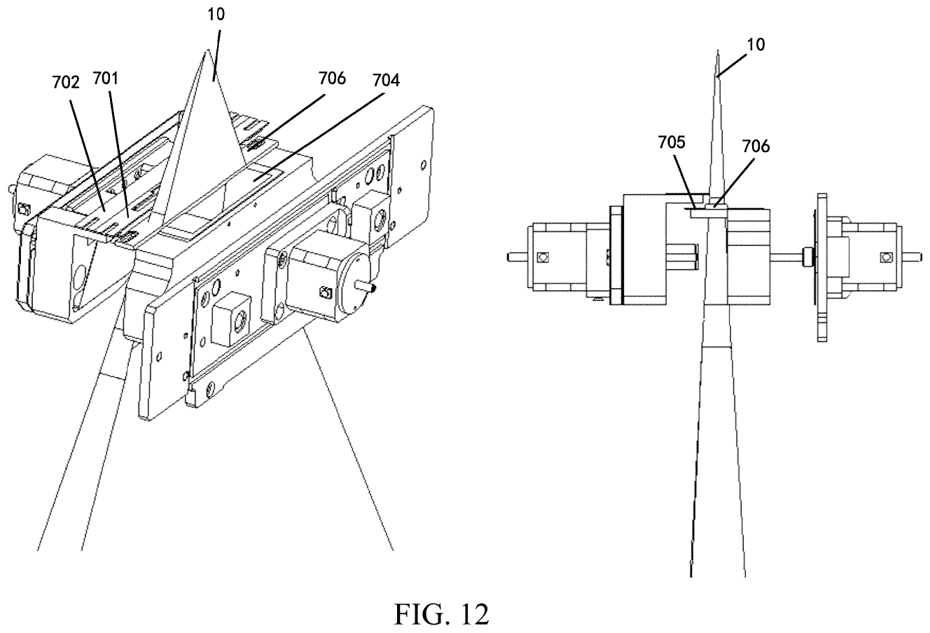

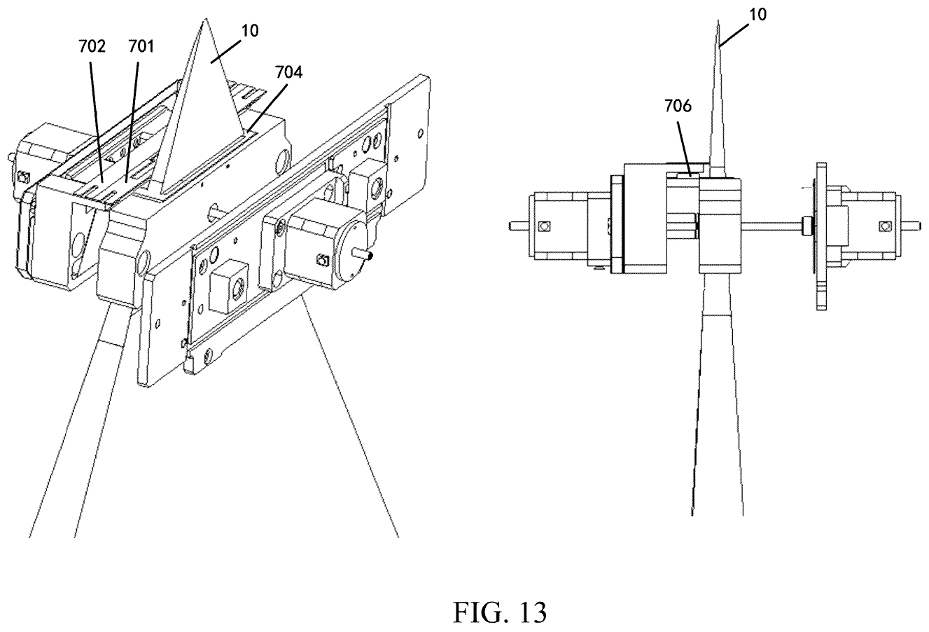

FIGS. 1 and 2 show exemplary embodiments of an imaging system, in which an apparatus for an imaging system provided by an embodiment of the present disclosure is positioned between a radiation source and a scan subject. Although a CT system is described by way of example, it should be understood that the techniques of the present invention may also be useful when applied to images acquired using other imaging modalities (such as tomosynthesis, C-arm angiography, etc.). The discussion on CT imaging modalities in the present invention is provided only as an example of one suitable imaging modality.

Various embodiments may be implemented in conjunction with different types of imaging systems. For example, various embodiments may be implemented in conjunction with a CT imaging system, in which a radiation source projects a fan-shaped or cone-shaped beam. The fan-shaped or cone-shaped beam is collimated to be located in an x-y plane of a Cartesian coordinate system, and the plane is generally referred to as an “imaging plane”. The X-ray beam passes through a scan subject, such as a patient. The beam is irradiated on a radiation detector array after being attenuated by the scan subject. The intensity of the attenuated radiation beam received at the detector array depends on the attenuation of the X-ray beam by the scan subject. Each detector element of the array produces a separate electrical signal that is a measure of the intensity of the beam at the detector position. Intensity measurements from all detectors are separately acquired to generate a transmission profile.

In third-generation CT imaging systems, a gantry is used to rotate the radiation source and the detector array in the imaging plane around a subject to be imaged (such as a region of the scan subject), so that the angle at which the X-ray radiation beam intersects the imaged scan subject is constantly changing. A full gantry rotation occurs when the gantry completes a full 360-degree rotation. A set of X-ray attenuation measurements (e.g., projection data) from the detector array at one gantry angle is referred to as a “view”. Therefore, the view represents each incremental position of the gantry. A “scan” of the subject includes a set of views made at different gantry angles or viewing angles during one rotation of the X-ray source and detector.

In an axial diagnostic scan, projection data is processed to construct an image corresponding to a two-dimensional slice captured through the scan subject. A scout scan (also referred to herein as a positioning scan) provides a projection view along the longitudinal axis of the scan subject and typically provides aggregates, each of which includes the internal structure of the scan subject. A method for reconstructing an image according to a set of projection data is referred to in the art as a filtered back projection technique. The method includes converting attenuation measurements from a scan into integers referred to as “CT numbers” or “Hounsfield units” (HU), and these integers are used to control the brightness of corresponding pixels on a display.

Beam characteristics such as size, shape, and energy may be different for a scout scan (also referred to herein as a positioning scan) and a diagnostic scan. During certain scout and diagnostic scans, it is desirable to use a high-power X-ray source. The high power improves the quality of the diagnostic scan and improves the thermal stability of an X-ray tube including a target. However, an increase in X-ray power may increase the patient's exposure to X-ray radiation. A filter may be used in the path of the beam to attenuate the X-ray and reduce its energy before the X-ray beam enters the patient's body. It may be particularly desirable to use a plurality of filters for combined filtering during small beams (low beam coverage), while a single filter may be used for scans with large beam coverage. The plurality of filters may be mounted on separate brackets that can move in and out of the beam as needed. However, adding a plurality of brackets will increase the cost and complexity of the apparatus. Also, the time to complete the scan may be longer due to the need to move the brackets in and out of the beam between each part of the scan. Therefore, according to embodiments disclosed herein, the X-ray filtering apparatus may include filter assemblies arranged on two sides of the path of the radiation beam in a Z direction, and each filter assembly may include one or more filters. On the basis of scan settings, one or more filters from the two filter assemblies may be placed in the path of the radiation beam. At least one filter assembly is configured to include multiple layers of filters, and any layer among the multiple layers of filters may form a combined filter with a filter in the other filter assembly, so that the need for the number of filters (in particular bowtie filters) may be reduced, and thus, the cost and complexity of the apparatus may be reduced.

FIG. 1 shows an exemplary CT system 100 configured to allow fast and iterative image reconstruction. Specifically, the CT imaging system 100 is configured to image a scan subject 112 (such as a patient, an inanimate object, or one or more manufactured parts) and/or a foreign subject (such as a dental implant, a stent, and/or a contrast agent present in a body placed on a movable workbench 228). In one embodiment, the CT imaging system 100 includes a gantry 102, which in turn may further include at least one X-ray radiation source 104. The at least one X-ray radiation source is configured to project an X-ray radiation beam 106 for imaging the scan subject 112. The X-ray radiation source 104 includes an X-ray tube and a target. The X-ray tube generates X-rays by accelerating and focusing a high-energy electron beam onto the rotating target. When individual electrons strike the target, the energy released by the atomic interaction with the target isotropically generates X-ray photons in a polychromatic spectrum, the maximum energy of these X-ray photons matching the maximum energy of the incident electrons. The X-ray photons exit the tube through a window that defines the X-ray beam. The beam may then be collimated and adjusted by using collimator blades and one or more filters.

Specifically, the radiation source 104 is configured to project the X-ray 106 toward a detector array 108 positioned on the opposite side of the gantry 102. Although FIG. 1 depicts only a single radiation source 104, in certain embodiments, a plurality of radiation sources may be used to project a plurality of X-rays 106, so as to acquire projection data corresponding to the scan subject 112 at different energy levels. The radiation source may include an X-ray target made of graphite and metal.

In some embodiments, the CT imaging system 100 further includes an image processing unit 110, which is configured to reconstruct an image of a target volume of the scan subject 112 by using an iterative or analytical image reconstruction method. For example, the image processing unit 110 may reconstruct the image of the target volume of the scan subject 112 by using an analytical image reconstruction method such as filtered back projection (FBP). As another example, the image processing unit 110 may reconstruct the image of the target volume of the scan subject 112 by using an iterative image reconstruction method such as advanced statistical iterative reconstruction (ASIR), conjugate gradient (CG), maximum likelihood expectation maximization (MLEM), model-based iterative reconstruction (MBIR), or the like.

FIG. 2 shows an exemplary imaging system 200 similar to the CT system 100 in FIG. 1. According to an aspect of the present disclosure, the system 200 is configured to perform automatic exposure control in response to user input. In one embodiment, the imaging system 200 includes a detector array 108 (see FIG. 1). The detector array 108 further includes a plurality of detector elements 202, which together sense an X-ray beam 106 (see FIG. 1) passing through a scan subject 204 (such as a patient) to acquire corresponding projection data. Therefore, in one embodiment, the detector array 108 is fabricated in a multi-slice configuration including a plurality of rows of units or detector elements 202. In such configurations, one or more additional rows of detector elements 202 are arranged in a parallel configuration for acquiring projection data.

A filter apparatus 240 may be mounted in a gantry 102 between a radiation source 104 and the scan subject 204. The filter apparatus 240 may include filter assemblies (which will be described in detail below with reference to FIGS. 3-13) arranged on two sides of the path of the radiation beam in a Z direction. Each filter assembly may travel in and out of the beam in the z-direction when the beam is substantially in a y-direction. At least one filter assembly in the filter apparatus 240 may include multiple layers of filters. For example, two layers of filters, i.e., an upper layer of filters 241 and a lower layer of filters 242, are shown in FIG. 2. The lower layer of filters 242 may include a bowtie filter. The bowtie filter is shown here as a rectangular diagram as a schematic diagram, not an actual form. The bowtie filter is rigid and non-deformable. The bowtie filter may alternatively have different shapes and material configurations to provide appropriate X-ray-specific spectra to image various types of anatomical structures. The upper layer of filters 241 may include a functional filter having specific filtering characteristics. The upper layers of filters 241 and the lower layer of filters 242 may belong to the same filter assembly (that is, relative positions thereof cannot be changed) and be offset in the Z direction. In this way, either one of the upper layer of filters 241 and the lower layer of filters 242 may be moved into the path of the radiation beam in the Z direction to filter the radiation beam. The upper layer of filters 241 and the lower layer of filters 242 may also belong to different filter assemblies (that is, they may move relative to each other). In this case, one or both of the upper layer of filters 241 and the lower layer of filters 242 may be separately moved into the path of the radiation beam in the Z direction to filter the radiation beam individually or in combination.

The bowtie filter may change the spatial distribution of the radiation beam in an axial plane of the scan subject (such as the patient). For example, the redistributed radiation beam may have high energy at the center of the scan subject and low energy at the periphery of the scan subject. The bowtie filter may be designed to image specific anatomical structures or parts, such as the head, chest, and abdomen, of a human body. During imaging, the bowtie filter or the filter combination may be selected on the basis of the anatomical structure of the scan subject to be scanned, and the selected filter or filter combination may be placed in the path of the radiation beam. The filter may be changed from one to the other in response to changes in the anatomical structure. On the basis of the nature of the scan, the filter assembly may be positioned such that the filter may or may not be placed in the path of the radiation beam. The filter may attenuate the beam and remove low energy components, thereby adjusting the beam for a particular scan, such as a scout scan.

A filter driving system may be coupled to the respective filter assemblies of the apparatus 240 to move one or more filters in and out of the path of the radiation beam. In an embodiment, a motor may couple the filter in a bracket by means of a shaft. The filter may be introduced into or removed from the beam path by translating the filter along an axis using a motor rotating shaft. One of the filters may be selected and translated into the X-ray beam between the radiation source and the scan subject to image a particular part of the human body. A computing device 216 may send a command to the motor of the filter driving system to move the selected filter into the radiation beam. The filter driving system may also send position information of the filter back to the computing device 216.

In certain embodiments, the system 200 is configured to traverse different angular positions around the scan subject 204 to acquire desired projection data. Therefore, the gantry 102 and components (such as the radiation source 104, the filter apparatus 240, and the detector 202) mounted thereon can be configured to rotate about a center of rotation 206 to acquire, for example, projection data at different energy levels. Alternatively, in embodiments in which the projection angle with respect to the scan subject 204 changes over time, the mounted components may be configured to move along a substantially curved line rather than a segment of a circumference.

In one embodiment, the system 200 includes a control mechanism 208 to control movement of the components, such as the rotation of the gantry 102 and the operation of the X-ray radiation source 104. In certain embodiments, the control mechanism 208 further includes an X-ray controller 210, which is configured to provide power and timing signals to the radiation source 104. Additionally, the control mechanism 208 includes a gantry motor controller 212, configured to control the rotational speed and/or position of the gantry 102 on the basis of imaging requirements.

In certain embodiments, the control mechanism 208 further includes a data acquisition system (DAS) 214, which is configured to sample analog data received from the detector elements 202, and to convert the analog data into digital signals for subsequent processing. The data sampled and digitized by the DAS 214 is transmitted to the computing device (also referred to as a processor) 216. In one example, the computing device 216 stores data in a storage device 218. For example, the storage device 218 may include a hard disk drive, a floppy disk drive, a compact disc-read/write (CD-R/W) drive, a digital versatile disc (DVD) drive, a flash drive, and/or a solid-state storage device.

Additionally, the computing device 216 provides commands and parameters to one or more of the DAS 214, the X-ray controller 210, and the gantry motor controller 212 to control system operations, such as data acquisition and/or processing. In certain embodiments, the computing device 216 controls system operations on the basis of operator input. The computing device 216 receives the operator input by means of an operator console 220 that is operably coupled to the computing device 216, the operator input including, for example, commands and/or scan parameters. The operator console 220 may include a keyboard or a touch screen to allow the operator to specify commands and/or scan parameters.

Although FIG. 2 shows only one operator console 220, more than one operator console may be coupled to the system 200, and, for example, is used to input or output system parameters, request examination, and/or view images. Moreover, in some embodiments, the system 200 may be coupled to, for example, a plurality of displays, printers, workstations, and/or similar devices located locally or remotely within an institution or hospital or in a completely different location by means of one or more configurable wired and/or wireless networks (such as the Internet and/or a virtual private network).

For example, in one embodiment, the system 200 includes or is coupled to a picture archiving and communication system (PACS) 224. In one exemplary embodiment, the PACS 224 is further coupled to a remote system (such as a radiology information system or a hospital information system), and/or an internal or external network (not shown) to allow operators in different locations to provide commands and parameters and/or acquire access to image data.

The computing device 216 uses operator-provided and/or system-defined commands and parameters to operate a workbench motor controller 226, which can in turn control a motorized workbench 228. Specifically, the workbench motor controller 226 moves the workbench 228 to properly position the scan subject 204 in the gantry 102 to acquire projection data corresponding to a target volume of the scan subject 204.

As described previously, the DAS 214 samples and digitizes the projection data acquired by the detector elements 202. Subsequently, an image reconstructor 230 uses the sampled and digitized X-ray data to perform high-speed reconstruction. Although the image reconstructor 230 is shown as a separate entity in FIG. 2, in certain embodiments, the image reconstructor 230 may form a part of the computing device 216. Alternatively, the image reconstructor 230 may not be present in the system 200, and the computing device 216 may instead perform one or more functions of the image reconstructor 230. In addition, the image reconstructor 230 may be located locally or remotely, and the image reconstructor 230 may be operably connected to the system 100 by using a wired or wireless network. Specifically, in one exemplary embodiment, computing resources in a “cloud” network cluster may be used for the image reconstructor 230.

In one embodiment, the image reconstructor 230 stores a reconstructed image in the storage device 218. Alternatively, the image reconstructor 230 transmits a reconstructed image to the computing device 216 to generate usable patient information for diagnosis and evaluation. In certain embodiments, the computing device 216 transmits a reconstructed image and/or patient information to a display 232, the display being communicatively coupled to the computing device 216 and/or the image reconstructor 230.

The present invention provides an apparatus for an imaging system. The apparatus includes: a first filter assembly, including at least one filter and located on one side of the path of a radiation beam; a second filter assembly, including at least one filter and located on the other side of the path of the radiation beam; and a driving system, used for moving the first filter assembly and the second filter assembly relative to each other to move one filter in the first filter assembly and/or one filter in the second filter assembly into the path of the radiation beam; wherein in a path direction of the radiation beam, the first filter assembly includes at least a first layer of filters and a second layer of filters which are located at different heights, and the second filter assembly includes at least a third layer of filters, the third layer of filters being located at a different height than the first layer of filters or the second layer of filters. Since the third layer of filters and the first layer of filters or the second layer of filters are located at different heights, the apparatus of the present invention allows a plurality of different combined filters to be formed by combining the filters in the first layer of filters or the second layer of filters with the filters in the third layer of filters to achieve a plurality of X-ray filtering functions.

Referring to FIGS. 3A-3E, FIGS. 3A-3E are schematic block diagrams of an apparatus 300 for an imaging system according to a first exemplary embodiment of the present invention. As shown in FIG. 3A, the apparatus 300 for the imaging system may include a first filter assembly 310, a second filter assembly 320, and a driving system 330.

The first filter assembly 310 may include at least one filter and be located on one side of the path of a radiation beam 10 (shown on the left side of the path of the radiation beam 10 in FIG. 3A). In a path direction (i.e., a Y-direction) of the radiation beam 10, the first filter assembly 310 may include at least a first layer of filters 311 and a second layer of filters 312 which are located at different heights. In the present invention, “the first layer of filters 311 and the second layer of filters 312 being located at different heights in the Y-direction “means that the first layer of filters 311 and the second layer of filters 312 are offset in the Y-direction, that is, the projections thereof on the Y-axis do not overlap.

The second filter assembly 320 may include at least one filter and be located on the other side of the path of the radiation beam 10 (shown on the right side of the path of the radiation beam 10 in FIG. 3A). The second filter assembly 320 may include at least a third layer of filters 323. In the path direction (i.e., the Y-direction) of the radiation beam 10, the third layer of filters 323 is located at a different height than the first layer of filters 311 or the second layer of filters 312. In other words, at least one of the first layer of filters 311 and the second layer of filters 312 is offset in the Y direction from the third layer of filters 323, that is, the projections thereof on the Y axis do not overlap.

The driving system 330 may be configured to move the first filter assembly 310 and the second filter assembly 320 relative to each other to move one filter in the first filter assembly 310 and/or one filter in the second filter assembly 320 into the path of the radiation beam 10. Specifically, the driving system 330 may be configured to move the first filter assembly 310 in the Z-direction such that the radiation beam 10 passes through one filter in the first filter assembly 310, and/or to move the second filter assembly 320 in the Z-direction such that the radiation beam 10 passes through one filter in the second filter assembly 320. The driving system 330 may independently move each of the first filter assembly 310 and the second filter assembly 320.

In some embodiments of the present invention, the third layer of filters 323 is located at a different height than at least the first layer of filters 311. The third layer of filters 323 and the second layer of filters 312 may be located at the same height, or may be located at different heights. In the first exemplary embodiment shown in FIG. 3A, the third layer of filters 323 and the second layer of filters 312 may be located at the same height, which means that the third layer of filters 323 and the second layer of filters 312 are not completely offset in the Y direction, that is, the projections thereof on the Y axis may partially or completely overlap.

In the apparatus 300 for an imaging system of the present invention, since the third layer of filters 323 is located at a different height than the first layer of filters 311, a plurality of different combined filters can be formed by combining the filters in the first layer of filters with the filters in the third layer of filters to implement a plurality of X-ray filtering functions.

In some embodiments of the present invention, the first layer of filters 311 may include at least a first filter 301 having a first filtering characteristic and a second filter 302 having a second filtering characteristic, the second layer of filters 312 may include at least a third filter 303 having a third filtering characteristic, and the third layer of filters 323 may include at least a fourth filter 304 having a fourth filtering characteristic.

In the first exemplary embodiment, the fourth filter 304 may be a bowtie filter. Since the third layer of filters 323 (including the fourth filter 304) is located at a different height than the first layer of filters 311 (including the first filter 301) in the Y-direction, the first filter 301 and the fourth filter 304 can be simultaneously moved into the path of the radiation beam 10 to form a filter combination, as shown in FIG. 3B.

In some embodiments of the present invention, the first filter 301 may be an adult head filter such that the filter combination is adapted to filter the radiation beam 10 when imaging an adult's head. In this case, the fourth filter 304 may be a bowtie filter adapted to filter the radiation beam 10 when imaging a subject having a small body size or a child's head. In this way, imaging for anatomical structures of different morphologies can be achieved using a single bowtie filter, thereby reducing the number of bowtie filters.

In some embodiments of the present invention, the first filter 301 may be a shaping filter 601 having a cross-shaped hollow portion, as shown in FIG. 6. The radiation beam 10 may pass through the cross-shaped hollow portion 20 of the shaping filter 601 without being attenuated. A peripheral portion (a black region in FIG. 6) of the shaping filter 601 that defines the cross-shaped hollow portion 20 may be formed of a material (for example, a tungsten alloy) capable of attenuating the radiation beam 10. The shaping filter 601 may form a filter combination with a bowtie filter, thereby filtering the radiation beam 10 when performing imaging and scanning of a particular anatomical structure of the scan subject. The advantages of the shaping filter 601 are as follows: the radiation beam 10 is allowed to pass through the cross-shaped hollow portion 20 to perform imaging and scanning of the particular anatomical structure (e.g., heart) of the scan subject, while the peripheral portion may attenuate or absorb the radiation beam 10 to reduce the radiation dose reaching the patient, thereby reducing harm to the scan subject while ensuring required imaging and scanning of the particular anatomical structure of the scan subject is performed.

In some embodiments of the present invention, the fourth filter 304, which is adapted to be moved into the path of the radiation beam 10 simultaneously with the shaping filter 601 to form a filter combination, may be a bowtie filter adapted to filter the radiation beam 10 when imaging a subject having a large body size.

In some embodiments of the present invention, the second filter 302 may include a functional filter that can be used individually. As an example, the second filter 302 may be a transfer function correction (TFC) filter for performing a correction operation on the imaging system. As an example, the correction operation may be as follows: a radiation beam emitted from a tube passes through the TFC filter, a high absorption coefficient material (such as a tungsten alloy) on the TFC filter and its sharp edges are expected to leave a projection on the detector, and analysis of the sharpness of the projection boundary can be used to determine an optimization parameter for a tube focus run-out design. As another example, the second filter 302 may be a blocking filter through which the radiation beam 10 cannot penetrate.

In some embodiments of the present invention, one of the second filter 302, the third filter 303, and the fourth filter 304 may be moved into the path of the radiation beam 10 to filter the radiation beam 10, as shown in FIG. 3C, 3D, or 3E.

In some embodiments of the present invention, none of the filters in the first filter assembly 310 and the second filter assembly 320 may be located in the path of the radiation beam 10, so that there is a gap between the first filter assembly 310 and the second filter assembly 320 through which the radiation beam 10 directly passes, as shown in FIG. 3A.

In some embodiments of the present invention, the first layer of filters 311 and the second layer of filters 312 may not overlap in a fan beam width direction (a Z-direction) of the radiation beam 10, which means that the first layer of filters 311 and the second layer of filters 312 are completely offset in the Z direction, that is, the projections thereof on a Z-axis do not overlap.

In some embodiments of the present invention, the first filter 301 and the second filter 302 may be mounted in the same plane. In the Z-direction, the first filter 301 may be located between the second filter 302 and the path of the radiation beam 10, and between the third filter 303 and the path of the radiation beam 10. In other words, the first filter 301 may be closer to the path of the radiation beam 10 than the second filter 302 and the third filter 303, and thus may first be moved into the path of the radiation beam 10 to form a filter combination with the fourth filter 304. Although FIG. 3A shows that the second filter 302 is located between the first filter 301 and the third filter 303 in the Z-direction, the positions of the second filter 302 and the third filter 303 may be exchanged in the Z-direction.

Referring to FIGS. 4A-4C, FIGS. 4A-4C are schematic block diagrams of an apparatus 400 for an imaging system according to a second exemplary embodiment of the present invention. As shown in FIG. 4A, the apparatus 400 for the imaging system may include a first filter assembly 410, a second filter assembly 420, and a driving system 430.

The first filter assembly 410 may include at least one filter and be located on one side of the path of a radiation beam 10 (shown on the left side of the path of the radiation beam 10 in FIG. 4A). In a path direction (i.e., a Y-direction) of the radiation beam 10, the first filter assembly 410 may include at least a first layer of filters 411 and a second layer of filters 412 which are located at different heights. In the present invention, “the first layer of filters 411 and the second layer of filters 412 being located at different heights in the Y-direction” means that the first layer of filters 411 and the second layer of filters 412 are offset in the Y-direction, that is, the projections thereof on the Y-axis do not overlap.

The second filter assembly 420 may include at least one filter and be located on the other side of the path of the radiation beam 10 (shown on the right side of the path of the radiation beam 10 in FIG. 4A). The second filter assembly 420 may include at least a third layer of filters 423. In the path direction (i.e., the Y-direction) of the radiation beam 10, the third layer of filters 423 is located at a different height than the first layer of filters 411 and the second layer of filters 412. In other words, each of the first layer of filters 411 and the second layer of filters 412 is offset in the Y direction from the third layer of filters 423, that is, the projections thereof on the Y axis do not overlap.

The driving system 430 may be configured to move the first filter assembly 410 and the second filter assembly 420 relative to each other to move one filter in the first filter assembly 410 and/or one filter in the second filter assembly 420 into the path of the radiation beam 10. Specifically, the driving system 430 may be configured to move the first filter assembly 410 in the Z-direction such that the radiation beam 10 passes through one filter in the first filter assembly 410, and/or to move the second filter assembly 420 in the Z-direction such that the radiation beam 10 passes through one filter in the second filter assembly 420. The driving system 430 may independently move each of the first filter assembly 410 and the second filter assembly 420.

In the apparatus 400 for an imaging system of the present invention, since each of the first layer of filters 411 and the second layer of filters 412 is located at a different height than the third layer of filters 423, a plurality of different combined filters can be formed by combining the filters in the first layer of filters 411 or the second layer of filters 412 with the filters in the third layer of filters 423 to implement a plurality of X-ray filtering functions.

In some embodiments of the present invention, the third layer of filters 423 may be located between the first layer of filters 411 and the second layer of filters 412 in the path direction (i.e., the Y-direction) of the radiation beam 10, so that the third layer of filters 423 may be inserted between the first layer of filters 411 and the second layer of filters 412 when the first filter assembly 410 the first filter assembly 410 are moved relative to each other.

In some embodiments of the present invention, the first layer of filters 411 may include at least a first filter 401 having a first filtering characteristic and a second filter 402 having a second filtering characteristic, the second layer of filters 412 may include at least a third filter 403 having a third filtering characteristic, and the third layer of filters 423 may include at least a fourth filter 404 having a fourth filtering characteristic and a fifth filter 405 having a fifth filtering characteristic. The fourth filter 404 and the fifth filter 405 may not overlap in a fan beam width direction (a Z-direction) of the radiation beam 10, which means that the fourth filter 404 and the fifth filter 405 are completely offset in the Z-direction, that is, the projections thereof on a Z axis do not overlap.

In the second exemplary embodiment, since the third layer of filters 423 (including the fourth filter 404) is located at a different height than the third layer of filters 412 (including the third filter 403) in the Y-direction, the third filter 403 and the fourth filtering 404 can be simultaneously moved into the path of the radiation beam 10 to form a filter combination, as shown in FIG. 4B.

In some embodiments of the present invention, the fourth filter 404 may be an adult head filter such that the filter combination is adapted to filter the radiation beam 10 when imaging an adult's head. In this case, the third filter 403 may be a bowtie filter adapted to filter the radiation beam 10 when imaging a subject having a small body size or a child's head. In this way, imaging for anatomical structures of different morphologies can be achieved using a single bowtie filter, thereby reducing the number of bowtie filters.

In some embodiments of the present invention, the fourth filter 404 may be a shaping filter 601 having a cross-shaped hollow portion, as shown in FIG. 6. The radiation beam 10 may pass through the cross-shaped hollow portion 20 of the shaping filter 601 without being attenuated. A peripheral portion (a black region in FIG. 6) of the shaping filter 601 that defines the cross-shaped hollow portion 20 may be formed of a material (for example, a tungsten alloy) capable of attenuating the radiation beam 10. The shaping filter 601 may form a filter combination with a bowtie filter, thereby filtering the radiation beam 10 when performing imaging and scanning of a particular anatomical structure of the scan subject. The advantages of the shaping filter 601 are as follows: the radiation beam 10 is allowed to pass through the cross-shaped hollow portion 20 to perform imaging and scanning of the particular anatomical structure (e.g., heart) of the scan subject, while the peripheral portion may attenuate or absorb the radiation beam 10 to reduce the radiation dose reaching the patient, thereby reducing harm to the scan subject while ensuring required imaging and scanning of the particular anatomical structure of the scan subject is performed.

In some embodiments of the present invention, the third filter 403, which is adapted to be moved into the path of the radiation beam 10 simultaneously with the shaping filter 601 to form a filter combination, may be a bowtie filter adapted to filter the radiation beam 10 when imaging a subject having a large body size.

In some embodiments of the present invention, the fifth filter 405 may include a functional filter that can be used individually. As an example, the fifth filter 405 may be a transfer function correction (TFC) filter for performing a correction operation on the imaging system. As an example, the correction operation may be as follows: a radiation beam emitted from a tube passes through the TFC filter, a high absorption coefficient material (such as a tungsten alloy) on the TFC filter and its sharp edges are expected to leave a projection on the detector, and analysis of the sharpness of the projection boundary can be used to determine an optimization parameter for a tube focus run-out design. As another example, the fifth filter 405 may be a blocking filter through which the radiation beam 10 cannot penetrate. The fifth filter 405 may be moved into the path of the radiation beam 10 to filter the radiation beam 10, as shown in FIG. 4C.

In some embodiments of the present invention, one of the first filter 401, the second filter 402, and the third filter 403 may be individually moved into the path of the radiation beam 10 to filter the radiation beam 10.

In some embodiments of the present invention, the first layer of filters 411 and the second layer of filters 412 may not overlap in the fan beam width direction (the Z-direction) of the radiation beam 10, which means that the first layer of filters 411 and the second layer of filters 412 are completely offset in the Z-direction, that is, the projections thereof on the Z axis do not overlap.

In some embodiments of the present invention, the fourth filter 404 and the fifth filter 405 may be mounted in the same plane. In the Z-direction, the fourth filter 404 may be located between the fifth filter 405 and the path of the radiation beam 10. In other words, the fourth filter 404 may be closer to the path of the radiation beam 10 than the fifth filter 405, and thus may first be moved into the path of the radiation beam 10 to form a filter combination with the third filter 403.

Referring to FIG. 5, FIG. 5 is a schematic block diagram of a X-ray filtering apparatus 500 for an imaging system according to a third exemplary embodiment of the present invention. Several details of the X-ray filtering apparatus 500 are the same as those of the apparatus 300 or 400 described above with respect to FIGS. 3-4, and will not be described again here. Special features of the X-ray filtering apparatus 500 will be mainly described below. As shown in FIG. 5, the X-ray filtering apparatus 500 for an imaging system may include a first filter assembly 510, a second filter assembly 520, and a driving system 530.

The first filter assembly 510 may include at least one filter and be located on one side of the path of the radiation beam 10. In the path direction (i.e., the Y-direction) of the radiation beam 10, the first filter assembly 510 may include at least a first upper layer of filters 511 and a first lower layer of filters 512 which are located at different heights. The first upper layer of filters 511 may include at least a first filter 501 having a first filtering characteristic and a second filter 502 having a second filtering characteristic, and the first lower layer of filters 512 may include at least a third filter 503 having a third filtering characteristic. The first filter 501, the second filter 502 and the third filter 503 may not overlap in the fan beam width direction (the Z direction) of the radiation beam 10.

The second filter assembly 520 may include at least one filter and be located on the other side of the path of the radiation beam 10. The second filter assembly 520 may include a second upper layer of filters 523 and/or a second lower layer of filters 524. The second lower layer of filters 524 may include a fourth filter 504 having a fourth filtering characteristic. The second upper layer of filters 523 may include a fifth filter 505 having a fifth filtering characteristic. The fourth filter 504 and the fifth filter 505 may not overlap in the fan beam width direction (the Z-direction) of the radiation beam 10.

In the path direction (i.e., the Y-direction) of the radiation beam 10, the second upper layer of filters 523 (including the fifth filter 505) is located at a different height than first upper layer of filters 511 and the first lower layer of filters 512 (including the third filter 503). In this way, the fifth filter 505 can be located together with the third filter 503 on the path of the radiation beam 10 to form a first combined filter.

In the path direction (i.e., the Y-direction) of the radiation beam 10, the second lower layer of filters 524 (including the fourth filter 504) is located at a different height than at least the first upper layer of filters 511 (including the first filter 501 and the second filter 502). In this way, the fourth filter 504 can be located together with at least one of the first filter 501 or the second filter 502 on the path of the radiation beam 10 to form a second combined filter.

In the X-ray filtering apparatus 500 for an imaging system of the present invention, since the first filter assembly 510 and the second filter assembly 520, which are arranged opposite to each other on two sides of the path of the radiation beam 10, have multiple layers of filters located at different heights, a plurality of different combined filters can be formed by combining the multiple layers of filters from the different filter assemblies to implement a plurality of X-ray filtering functions.

In some embodiments of the present invention, the second upper layer of filters 523 may further include a sixth filter 506 having a sixth filtering characteristic. The fourth filter 504, the fifth filter 505 and the sixth filter 506 may not overlap in the fan beam width direction (the Z direction) of the radiation beam 10.

The driving system 530 may be configured to move the first filter assembly 510 and the second filter assembly 520 relative to each other to move one filter (501/502/503) in the first filter assembly 510 and/or one filter (504/505/506) in the second filter assembly 520 into the path of the radiation beam 10.

In some embodiments of the present invention, the fifth filter 505 may be an adult head filter, so that the first filter combination (fifth filter 505+third filter 503) is adapted to filter the radiation beam 10 when imaging an adult's head. The third filter 503 may be a bowtie filter adapted to filter the radiation beam 10 when imaging a subject having a small body size or a child's head. The first filter 501 may be a shaping filter 601 having a cross-shaped hollow portion. The fourth filter 504, which is adapted to be moved into the path of the radiation beam 10 simultaneously with the shaping filter 601 to form a second filter combination, may be a bowtie filter adapted to filter the radiation beam 10 when imaging a subject having a large body size. In this way, imaging for anatomical structures of different morphologies can be achieved using a single bowtie filter, thereby reducing the number of bowtie filters.

In some embodiments of the present invention, the fifth filter 505 may be a shaping filter 601 having a cross-shaped hollow portion. The third filter 503, which is adapted to be moved into the path of the radiation beam 10 simultaneously with the shaping filter 601 to form a first filter combination, may be a bowtie filter adapted to filter the radiation beam 10 when imaging a subject having a large body size. The first filter 501 may be an adult head filter such that the second filter combination (first filter 501+fourth filter 504) is adapted to filter the radiation beam 10 when imaging an adult's head. The fourth filter 304 may be a bowtie filter adapted to filter the radiation beam 10 when imaging a subject having a small body size or a child's head.

In some embodiments of the present invention, the second filter 502 or the sixth filter 507 may include a functional filter that can be used individually. As an example, the second filter 502 may be a transfer function correction (TFC) filter and the sixth filter 507 may be a blocking filter through which the radiation beam 10 cannot penetrate. As another example, the second filter 502 may be a blocking filter through which the radiation beam 10 cannot penetrate, and the sixth filter 506 may be a transfer function correction (TFC) filter.

In some embodiments of the present invention, the first filter 501 and the second filter 502 may be mounted in the same plane. In the Z-direction, the first filter 501 may be located between the second filter 502 and the path of the radiation beam 10, and between the third filter 503 and the path of the radiation beam 10. In other words, the first filter 501 may be closer to the path of the radiation beam 10 than the second filter 502 and the third filter 503, and thus may first be moved into the path of the radiation beam 10 to form a filter combination with the fourth filter 504.

In some embodiments of the present invention, the fifth filter 505 and the sixth filter 506 may be mounted in the same plane. In the Z-direction, the fifth filter 505 may be located between the sixth filter 506 and the path of the radiation beam 10, and between the fourth filter 504 and the path of the radiation beam 10. In other words, the fifth filter 505 may be closer to the path of the radiation beam 10 than the sixth filter 506 and the fourth filter 504, and thus may first be moved into the path of the radiation beam 10 to form a filter combination with the third filter 503.

Referring to FIG. 7, FIG. 7 is FIG. 7 is an exemplary structure of a X-ray filtering apparatus 700 constructed according to the third exemplary embodiment of the present invention, wherein an oblique view of the X-ray filtering apparatus 700 is shown on the left side of FIG. 7, and a side view of the X-ray filtering apparatus 700 is shown on the right side of FIG. 7. Several details of the X-ray filtering apparatus 700 are the same as those of the apparatus 300, 400, or 500 described above with respect to FIGS. 3-5, and will not be described again here. As shown in FIG. 7, the X-ray filtering apparatus 700 may include a first filter assembly 710, a second filter assembly 720, and a driving system.

The first filter assembly 710 may include a first filter 701, a second filter 702, and a third filter 703. In the path direction (i.e., the Y-direction) of the radiation beam 10, the first filter 701 and the second filter 702 may be mounted in the same plane and located at a different height than the third filter 703. The first filter 701, the second filter 702, and the third filter 703 do not overlap in the fan beam width direction (the Z-direction) of the radiation beam 10. As an example, the first filter 701 is a shaping filter having a cross-shaped hollow portion, the second filter 702 is a blocking filter through which the radiation beam 10 cannot penetrate, and the third filter 703 is a first bowtie filter. The first bowtie filter is adapted to filter the radiation beam 10 when imaging a subject having a small body size or a child's head.

The second filter assembly 720 may include a fourth filter 704, a fifth filter 705, and a sixth filter 706. In the path direction (i.e., the Y-direction) of the radiation beam 10, the fifth filter 705 and the sixth filter 706 may be mounted in the same plane, located in a different plane than the first filter 501 and the second filter 502, and located at a different height than the fourth filter 704. The fourth filter 704, the fifth filter 705, and the sixth filter 706 do not overlap in the fan beam width direction (the Z-direction) of the radiation beam 10. As an example, the fourth filter 704 is a second bowtie filter, the fifth filter 705 is an adult head filter, and the sixth filter 706 is a TFC filter. The second bowtie filter is adapted to filter the radiation beam 10 when imaging a subject having a large body size.

The first filter assembly 710 may have a first holder. The first holder has a first main body 711, and the first main body 711 has a cavity for accommodating the third filter 703. Directly above the third filter 703, the first main body 711 has a hollowed-out structure. The first holder further has a plurality of first suspension arms 712, and each first suspension arm 712 extends from two ends of the first main body 711 toward the second filter assembly 720 to support the first filter 703 and the second filter 704.

The X-ray filtering apparatus 700 may further include a first stationary component 740. The driving system may include a first motor 731 and a first shaft 732. The first motor 731 is fixed to the first stationary component 740. The first shaft 732 is coupled to the first motor 731 and the first filter assembly 710. The first stationary component 740 may be fixed to a gantry of an imaging system to remain stationary. The first filter assembly 710 may be moved between a plurality of positions by actuating the first motor 731, so as to move one of the first filter 701, the second filter 702, and the third filter 703 into or out of the path of radiation beam 10.

The second filter assembly 720 may have a second holder. The second holder has a second main body 721, and the second main body 721 has a cavity for accommodating the fourth filter 704. Directly above the fourth filter 704, the second main body 721 has a hollowed-out structure. The second holder further has a plurality of second suspension arms 722, and each second suspension arm 722 extends from two ends of the second main body 721 toward the first filter assembly 710 to support the fifth filter 705 and the sixth filter 706.

The X-ray filtering apparatus 700 may further include a second stationary component 750. The driving system may further include a second motor 733 and a second shaft 734. The second motor 733 is fixed to the second stationary member 750. The second shaft 734 is coupled to the second motor 733 and the second filter assembly 720. The second stationary component 750 may be fixed to the gantry of the imaging system to remain stationary. The second filter assembly 720 may be moved between a plurality of positions by actuating the second motor 733 to move one of the fourth filter 704, the fifth filter 705, and the sixth filter 706 into or out of the path of the radiation beam 10.

Referring to FIGS. 8-13, examples of various working positions of the X-ray filtering apparatus 700 are shown.

In the example of FIG. 8, the third filter 703 is moved into the path of the radiation beam 10, and only the third filter 703 is used to filter the radiation beam 10, implementing a first X-ray filtering function. The first X-ray filtering function is adapted for a process of imaging a subject having a small body size or a child's head.

In the example of FIG. 9, the second filter 702 is moved into the path of the radiation beam 10, and only the third filter 702 is used to filter the radiation beam 10, implementing a second X-ray filtering function. The second X-ray filtering function is suitable for a scenario in which the radiation beam 10 needs to reach a scanning region without passing through the apparatus 700.

In the example of FIG. 10, the first filter 701 and the fourth filter 704 are moved into the path of the radiation beam 10, and a first filter combination formed by the first filter 701 and the fourth filter 704 is used to filter the radiation beam 10, implementing a third X-ray filtering function. The third X-ray filtering function is suitable for a process of imaging a particular anatomical structure that requires the radiation beam 10 to be shaped into a cross shape.

In the example of FIG. 11, the third filter 703 and the fifth filter 705 are moved into the path of the radiation beam 10, and a second filter combination formed by the third filter 703 and the fifth filter 705 is used to filter the radiation beam 10, implementing a fourth X-ray filtering function. The fourth X-ray filtering function is suitable for a process of imaging an adult's head.

In the example of FIG. 12, the sixth filter 706 is moved into the path of the radiation beam 10, and only the sixth filter 706 is used to filter the radiation beam 10, implementing a fifth X-ray filtering function. The fifth X-ray filtering function is suitable for a transfer function correction process performed on an imaging system.

In the example of FIG. 13, the fourth filter 704 is moved into the path of the radiation beam 10, and only the fourth filter 704 is used to filter the radiation beam 10, implementing a sixth X-ray filtering function. The sixth X-ray filtering function is suitable for a process of imaging a subject having a large body size.

As can be seen from FIGS. 7-13, the X-ray filtering apparatus 700 of the present invention has the following features: the first filter assembly 710 and the second filter assembly 720 are arranged opposite to each other on two sides of the path of the radiation beam 10 and each include double layers of filters; the first filter assembly 710 and the second filter assembly 720 each have four working positions (a position in which no filter is located in the path of the radiation beam 10 and positions in which three filters are each located in the path of the radiation beam 10); the third filter 703 for filtering the radiation beam 10 when imaging a subject having a small body size or a child's head is arranged on the same side as the first filter 701 for shaping the radiation beam 10 into a cross shape, and they are separated by one filter 702 in the middle; the adult head filter 705 is designed on the outermost portion of the second filter assembly 720 on the opposite side of the first filter assembly 710 (including the third filter 703); the third filter 703 and the fourth filter 704, which are closest to the driving motors, are each designed as a structure which is hollowed-out in an upper portion; and at least seven (the above-mentioned first to sixth X-ray filtering functions and the filter-free radiation beam pass-through function) X-ray filtering operation functions can be implemented.

The present invention further provides a corresponding imaging system.

The imaging system (e.g., the imaging system 100 or 200 in FIG. 1 or 2) may perform imaging examination on the basis of a scanning protocol. The imaging system may include a gantry 102, a radiation source 104, a detector 108, a motorized workbench 228, the apparatus 300/400/500/700 described above, and a controller. The controller may be configured to control the driving system on the basis of the scanning protocol such that a filter in the first filter assembly and/or the second filter assembly that conforms to the scanning protocol is moved into the path of the radiation beam.

So far, the X-ray filtering apparatus according to the present invention and the imaging system including the same have been described. In the X-ray filtering apparatus of the present invention, since the first filter assembly and the second filter assembly, which are arranged opposite to each other on two sides of the path of the radiation beam, have multiple layers of filters located at different heights, a plurality of different combined filters can be formed by combining the multiple layers of filters from the different filter assemblies to implement a plurality of X-ray filtering functions, thereby achieving different clinical and calibration purposes. In addition, the apparatus can reduce the number of bowtie filters required by combining the filters, thereby saving costs and apparatus volume. Meanwhile, the apparatus can have a compact layout in the Z direction, saving space, and facilitating adaptation to various existing or future imaging systems on the market.

Some exemplary embodiments have been described above. However, it should be understood that various modifications can be made to the exemplary embodiments described above without departing from the spirit and scope of the present invention. For example, an appropriate result can be achieved if the described techniques are performed in a different order and/or if the components of the described system, architecture, device, or circuit are combined in a different manner and/or replaced or supplemented with additional components or equivalents thereof. Accordingly, the modified other embodiments also fall within the protection scope of the claims.

1. An apparatus for an imaging system, comprising:
a first filter assembly including at least one filter and located on one side of the path of a radiation beam;
a second filter assembly including at least one filter and located on the other side of the path of the radiation beam; and
a driving system used for moving the first filter assembly and the second filter assembly relative to each other to move one filter in the first filter assembly and/or one filter in the second filter assembly into the path of the radiation beam;
wherein in a path direction of the radiation beam, the first filter assembly includes at least a first layer of filters and a second layer of filters located at different heights, and the second filter assembly includes at least a third layer of filters, the third layer of filters being located at a different height than the first layer of filters.

2. The apparatus according to claim 1, wherein the first layer of filters includes at least a first filter having a first filtering characteristic and a second filter having a second filtering characteristic, the second layer of filters includes at least a third filter having a third filtering characteristic, and the third layer of filters includes at least a fourth filter having a fourth filtering characteristic.

3. The apparatus according to claim 2, wherein the first filter and the fourth filter are capable of being simultaneously moved into the path of the radiation beam to form a filter combination, wherein the fourth filter is a bowtie filter.

4. The apparatus according to claim 3, wherein the first filter is an adult head filter such that the filter combination is adapted to filter the radiation beam when imaging an adult's head.

5. The apparatus according to claim 2, wherein the first filter is a shaping filter having a cross-shaped hollow portion, and the radiation beam passes through the cross-shaped hollow portion of the first filter without being attenuated.

6. The apparatus according to claim 2, wherein the second filter includes a transfer function correction filter or a blocking filter through which the radiation beam cannot penetrate.

7. The apparatus according to claim 1, wherein the first layer of filters and the second layer of filters do not overlap in a fan beam width direction of the radiation beam.

8. The apparatus according to claim 2, wherein the second filter assembly includes a fourth layer of filters, and the fourth layer of filters is located at a different height than the first layer of filters in the path direction of the radiation beam.

9. The apparatus according to claim 8, wherein the fourth layer of filters includes at least one filter that does not overlap with the third layer of filters in a fan beam width direction of the radiation beam.

10. The apparatus according to claim 2, wherein the third layer of filters further includes a fifth filter having a fifth filtering characteristic, and the fifth filter does not overlap with the fourth filter in a fan beam width direction of the radiation beam.

11. The apparatus according to claim 2, wherein the third filter and the fourth filter are capable of being simultaneously moved into the path of the radiation beam to form a filter combination.

12. The apparatus according to claim 11, wherein the fourth filter is a shaping filter having a cross-shaped hollow portion, and the radiation beam passes through the cross-shaped hollow portion of the fourth filter without being attenuated.

13. An X-ray filtering apparatus for an imaging system, comprising:
a first filter assembly including at least one filter and located on one side of the path of a radiation beam;
a second filter assembly including at least one filter and located on the other side of the path of the radiation beam; and
a driving system used for moving the first filter assembly and the second filter assembly relative to each other to move one filter in the first filter assembly and/or one filter in the second filter assembly into the path of the radiation beam,
wherein in a path direction of the radiation beam, the first filter assembly includes at least a first layer of filters and a second layer of filters located at different heights, and the second filter assembly includes at least a third layer of filters, the third layer of filters being located at a different height than the first layer of filters;
the first layer of filters includes at least a first filter having a first filtering characteristic and a second filter having a second filtering characteristic;
the second layer of filters includes at least a third filter having a third filtering characteristic; and
the third layer of filters includes at least a fourth filter having a fourth filtering characteristic, and the fourth filter is capable of respectively forming a combined filter with at least one of the first filter, the second filter, or the third filter.

14. The apparatus according to claim 13, wherein the first filter is a shaping filter having a cross-shaped hollow portion, and the radiation beam passes through the cross-shaped hollow portion of the first filter without being attenuated.

15. The apparatus according to claim 13, wherein the first filter and the second filter are mounted in the same plane, and the first filter is located between the second filter and the X-ray radiation path.

16. The apparatus according to claim 14, wherein the first filter is located in the path of the radiation beam together with the fourth filter to form a first combined filter.

17. The apparatus according to claim 13, wherein the second filter assembly includes a fourth layer of filters, and the fourth layer of filters includes at least one sixth filter that does not overlap with the fourth filter in a fan beam width direction of the radiation beam.

18. The apparatus according to claim 17, wherein the sixth filter is capable of respectively forming a combined filter with at least one of the first filter, the second filter, or the third filter.

19. The apparatus according to claim 18, wherein the sixth filter is capable of being located in the path of the radiation beam together with the third filter to form a second combined filter.
